(19) United States
(12) Patent Application Publication
(10) Pub. No.: US 2006/0130597 A1
(43) Pub. Date: Jun. 22, 2006

(54) Container handling device

(76) Inventors: Christopher J. Bernard, Cheshire, CT (US); James J. Mongillo, Northford, CT (US); Moneesh Chatterjee, Madison, CT (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(21) Appl. No.: 11/271,602

(22) Filed: Nov. 10, 2005

Related U.S. Application Data
(60) Provisional application No. 60/628,770, filed on Nov. 17, 2004.

(51) Int. Cl. G01N 35/10; C12Q 1/68

(52) U.S. Cl. 73/864.21; 422/50

(57) ABSTRACT

A micro-tube handling device is provided and includes a lower cap-nest, wherein the lower cap-nest includes a rack cradle surrounding a rack nest opening, a tube support device, wherein the tube support device is disposed to be associated with the rack nest opening to allow the tube support device access to the lower cap-nest via the rack nest opening, a cap-lid device, wherein the cap-lid device includes a cap retainer portion disposed adjacent a plurality of a cap-lid cavities and a stripper block device, wherein the stripper block device includes a plurality of stripper block pins, wherein each of the plurality of stripper block pins are disposed to be aligned with each of the plurality of cap-lid cavities.

### CROSS-REFERENCE TO RELATED APPLICATIONS

This application claims the benefit of U.S. Provisional Application Ser. No. 60/628,770 filed Nov. 17, 2004.

### FIELD OF THE INVENTION

This disclosure relates generally to the handling of micro-tube vessels and more particularly to an apparatus for the capping and de-capping of micro-tube vessels.

### BACKGROUND OF THE INVENTION

A micro-tube is a small plastic or glass container which includes a tube inlet and defines a tube cavity for containing a reagent or other fluidic substance for processing which can be either in a wet or dry form. Once the reagent or other fluidic substance is introduced into the tube cavity via the tube inlet, usually with a micro-pipette, a tube cap is associated with the tube inlet to seal the reagent or other fluidic substance within the tube cavity. At this point, the reagent or other fluidic substance may be processed using a centrifuge or other processing device. Once the reagent has been processed, the reagent sample may be removed for analysis by uncapping the micro-tube and dispensing the reagent sample into a more suitable vessel.

Typically, micro-tubes are used in quantity and are usually contained in a micro-tube rack. One common rack has 96 wells arranged in eight rows of twelve, wherein each well may contain a single micro-tube. As can be appreciated, if the micro-tubes are empty then the act of uncapping does not present any problem of ejecting the contents. However, when the micro-tubes are full or partially full (as in the case of micro-tubes purchased or prepackaged with reagents), it is often the case that some of the contents of the micro-tubes will be inadvertently released during uncapping. Moreover, it is sometimes the case that recapping the micro-tubes might also result in the spillage of some of the contents of the tube cavity. This is highly undesirable for at least two reasons. First, there is a risk of exposing the technician to a substance that may be deleterious to the health of the technician. As such, universal precautions dictate that the reagent substance be contained in a controlled manner at all times. Second, any leakage or spillage of the reagent increases the risk of cross contamination, possibly causing adjacent and/or subsequent reactions to become contaminated. As such, each of the micro-tube caps need to be carefully removed and/or replaced one cap at a time. This is not only tedious and time consuming, but also requires repetitive movements.

One reason spillage tends to occur during the decapping and/or recapping process involves the size of the tubes and the quantities in which they are used. For example, consider a 96 well micro-tube rack, where each well contains one micro-tube. In order to keep the size of the rack to a minimum (as lab space is typically at a premium), the micro-tubes are closely packed together. However, due to the small size of the micro-tubes (hence the name “micro-tube”) and the fact that the processing of the reagents (such as centrifugation) may cause the cap to become more securely associated with the micro-tube the removal of the cap becomes quite difficult and may cause the technician to bump into adjacent tubes. If the adjacent tube that has been bumped by the technician has already gone through the uncapping process, any substances contained within the tube may be spilled. Additionally, the repetitiveness of the cap removal process may become very cumbersome and can be complicated by the need for the lab technician to wear gloves. As such, a careless or hurried technician may not always get all of the tubes properly recapped all of the time ultimately resulting in test failures or corrupted test data due to contamination and/or evaporation during heating.

### SUMMARY OF THE INVENTION

A micro-tube handling device is provided and includes a rack nest, wherein the rack nest includes a rack cradle surrounding a rack nest opening, a rack lifting device, wherein the rack lifting device is disposed to be associated with the rack nest opening to allow the rack lifting device access to the rack nest via the rack nest opening, a cap-lid device, wherein the cap-lid device includes a cap retainer portion disposed adjacent a plurality of a cap-lid cavities and a stripper block device, wherein the stripper block device includes a plurality of stripper block pins, wherein each of the plurality of stripper block pins are disposed to be aligned with each of the plurality of cap-lid cavities.

A method for implementing a micro-tube handling device is provided, wherein the method includes obtaining a micro-tube handling device, a micro-tube rack and a cap-lid device, wherein the micro-tube handling device includes a lower device portion movably associated with an upper device portion, and wherein the micro-tube rack includes at least one micro-tube having a micro-tube opening and wherein the cap-lid device includes at least one micro-tube cap, wherein the at least one micro-tube cap is frictionally associated with the cap-lid device, disposing the micro-tube rack within the lower device portion and the cap-lid device within the upper device portion, operating the micro-tube handling device to associate the micro-tube rack with the cap-lid device such that the at least one micro-tube cap is snugly contained within the micro-tube opening, disassociating the micro-tube rack from the cap-lid device such that the at least one micro-tube cap is disassociated from the cap-lid device and removing the micro-tube rack and the cap-lid device from the micro-tube handling device.

A micro-tube handling device is provided and includes at least one collet pin and at least one collet, wherein the at least one collet includes a sizably configurable collet tip having a barbed portion and wherein the at least one collet defines a collet pin cavity for receiving the at least one collet pin, wherein the sizably configurable collet tip is configurable between a first collet tip size and a second collet tip size by disposing the at least one collet pin within the collet pin cavity.

A method for implementing a micro-tube handling device is provided, wherein the method includes obtaining the micro-tube handling device, wherein the micro-tube handling device includes at least one collet having a collet tip, wherein the collet tip includes a collet barbed portion and is sizably configurable between a first tip size and a second tip size, associating a micro-tube rack adjacent the micro-tube handling device, wherein the micro-tube rack includes at least one micro-tube having a micro-tube cap which defines a cap cavity, positioning the at least one collet adjacent the at least one micro-tube such that the collet barbed portion is at least partially disposed within the micro-tube cap cavity, configuring the at least one collet tip between the first tip size and the second tip size to cause the collet barbed portion to securingly interact with the micro-tube cap, repositioning the at least one collet away from the at least one micro-tube to cause the micro-tube cap to be removed from the micro-tube cavity and removing the micro-tube cap from the collet tip portion.

A machine-readable computer program code is provided, the program code including instructions for causing a controller to implement method for implementing a micro-tube handling device, wherein the micro-tube handling device includes at least one collet having a collet tip, wherein the collet tip includes a collet barbed portion and is sizably configurable between a first tip size and a second tip size, wherein the method includes associating a micro-tube rack adjacent the micro-tube handling device, wherein the micro-tube rack includes at least one micro-tube having a micro-tube cap which defines a cap cavity, positioning the at least one collet adjacent the at least one micro-tube such that the collet barbed portion is at least partially disposed within the micro-tube cap cavity, configuring the at least one collet tip between the first tip size and the second tip size to cause the collet barbed portion to securingly interact with the micro-tube cap, repositioning the at least one collet away from the at least one micro-tube to cause the micro-tube cap to be removed from the micro-tube cavity and removing the micro-tube cap from the collet tip portion.

A machine-readable computer program code is provided, the program code including instructions for causing a controller to implement method for implementing a micro-tube handling device, wherein the micro-tube handling device includes a micro-tube rack, a cap-lid device and a lower device portion movably associated with an upper device portion and wherein the micro-tube rack includes at least one micro-tube having a micro-tube opening and wherein the cap-lid device includes at least one micro-tube cap, wherein the at least one micro-tube cap is frictionally associated with the cap-lid device, wherein the method includes disposing the micro-tube rack within the lower device portion and the cap-lid device within the upper device portion, operating the micro-tube handling device to associate the micro-tube rack with the cap-lid device such that the at least one micro-tube cap is snugly contained within the micro-tube opening, disassociating the micro-tube rack from the cap-lid device such that the at least one micro-tube cap is disassociated from the cap-lid device and removing the micro-tube rack and the cap-lid device from the micro-tube handling device.

### BRIEF DESCRIPTION OF DRAWINGS

The foregoing and other features and advantages of the present invention will be more fully understood from the following detailed description of illustrative embodiments, taken in conjunction with the accompanying drawings in which:

FIG. 1 is a side perspective view of a micro-tube handling device, in accordance with a first embodiment;

FIG. 2 is a front view of a base actuation device for the micro-tube handling device in FIG. 1;

FIG. 3 is a front view of a tube support device and a tube support device mating plate for the micro-tube handling device in FIG. 1;

FIG. 4 is a top down view of the tube support device and the tube support device mating plate in FIG. 3;

FIG. 5 is a front view of a lower nest actuation device for the micro-tube handling device in FIG. 1;

FIG. 6 is a top down view of a lower cap-nest for the micro-tube handling device in FIG. 1;

FIG. 7 is a side view of the lower cap-nest in FIG. 6;

FIG. 8 is a front view of an upper actuation device for the micro-tube handling device of FIG. 1;

FIG. 9 is a front view of an upper nest actuation device for the micro-tube handling device of FIG. 1;

FIG. 10 is a bottom up view of a cap-lid device for the micro-tube handling device of FIG. 1;

FIG. 11 is a side view of the cap-lid device of FIG. 10;

FIG. 12 is a top down view of the cap-lid device of FIG. 10;

FIG. 13 is a top down view of an upper cap-nest for the micro-tube handling device of FIG. 1;

FIG. 14 is a side view of the upper cap-nest of FIG. 13;

FIG. 15 is a top down view of a cap-stripper device for the micro-tube handling device of FIG. 1;

FIG. 16 is a side view of the cap-stripper device of FIG. 15;

FIG. 17 is a bottom up view of the cap-stripper device of FIG. 15;

FIG. 18 is a side view of the assembly of the cap-lid device, the upper cap-nest and the cap-stripper device for the micro-tube handling device of FIG. 1;

FIG. 19 is a side view of a stripper device and the stripping device mating plate for the micro-tube handling device of FIG. 1;

FIG. 20 is a bottom up view of the stripper device of FIG. 19;

FIG. 21 is a block diagram illustrating a method for implement the micro-tube handling device in FIG. 1;

FIG. 22 is a side perspective view of a micro-tube handling device, in accordance with a second embodiment;

FIG. 23 is a side view of a pin holder device and a plurality of collet pins, for the micro-tube handling device of FIG. 22;

FIG. 24 is a side view of a collet holder device and a plurality of collets, for the micro-tube handling device of FIG. 22;

FIG. 25 is a side view of a collet, for the micro-tube handling device of FIG. 22;

FIG. 26 is a front view of the collet tip for the collet in FIG. 25;

FIG. 27 is a side view of the collet tip for the collet in FIG. 25;

FIG. 28 is a side view of the collet tip for the collet in FIG. 25 associated with the collet pin in FIG. 23;

FIG. 29 is a block diagram illustrating a method for implement the micro-tube handling device in FIG. 22;

FIG. 30 is a side view of a micro-tube rack containing a plurality of micro-tubes;

FIG. 31 is a side view of a micro-tube disassociated with its micro-tube cap; and

FIG. 32 is a side view of the micro-tube in FIG. 31 associated with its micro-tube cap.

FIG. 33 is a side view of the collet tip for the collet in FIG. 25 associated with a micro-tube cap; and

FIG. 34 is a side view of the collet tip for the collet in FIG. 25 associated with the collet pin in FIG. 23 and the micro-tube cap in FIG. 32.

### DETAILED DESCRIPTION OF THE INVENTION

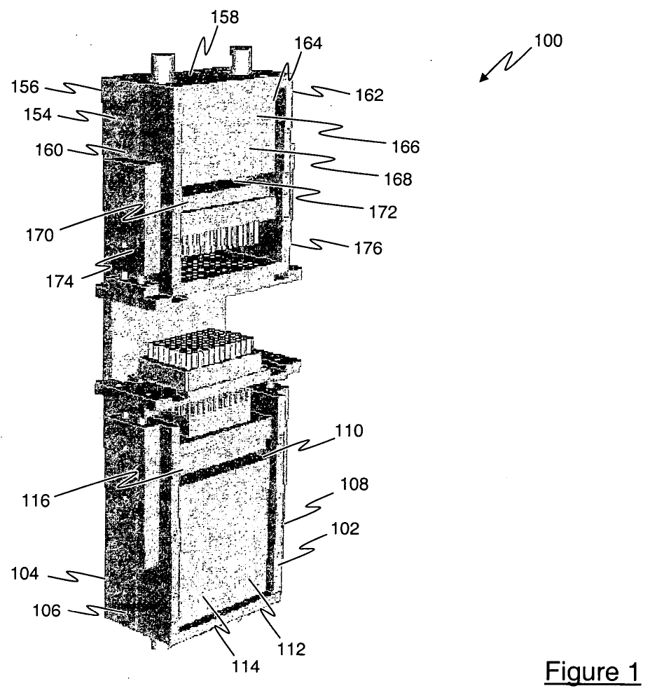

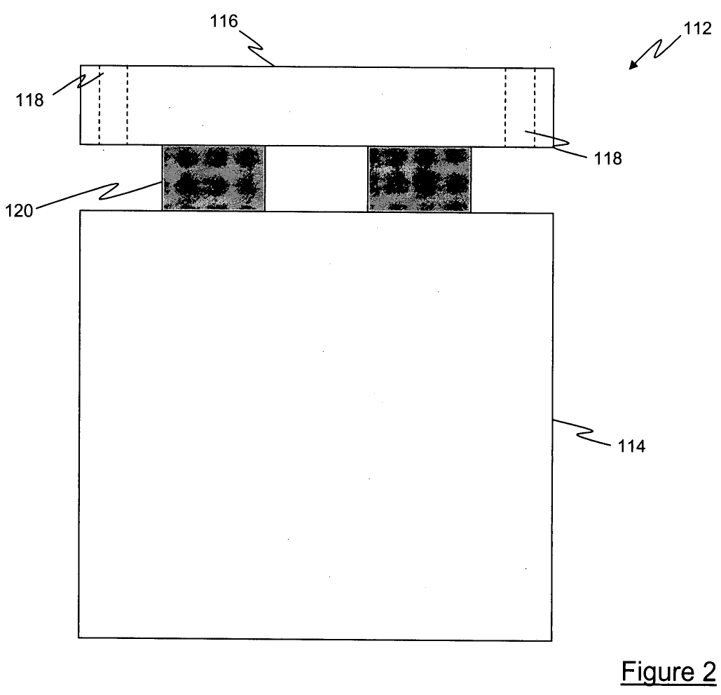

Referring to FIG. 1, a first embodiment of a micro-tube handling device 100 is shown and includes a base support structure 102 having a back support wall 104, a first side support wall 106 and a second side support wall 108, wherein back support wall 104, first side support wall 106 and second side support wall 108 define a cavity 110 for containing a base actuation device 112. The base actuation device 112 is non-movably disposed within cavity 110 via any method and/or device suitable to the desired end purpose, such as a bolt, screw, clip and/or adhesive. Referring to FIG. 2, base actuation device 112 includes a base actuator 114 and a base actuator interface plate 116, wherein the base actuator interface plate 116 includes at least one base actuator mounting hole 118 and is movably associated with base actuation device 112 via at least one base actuator piston 120. When the actuation device 112 is operated, the base actuator 114 causes the at least one base actuator piston 120, and hence the base actuator interface plate 116, to extend away from and/or contract toward the base actuator 114.

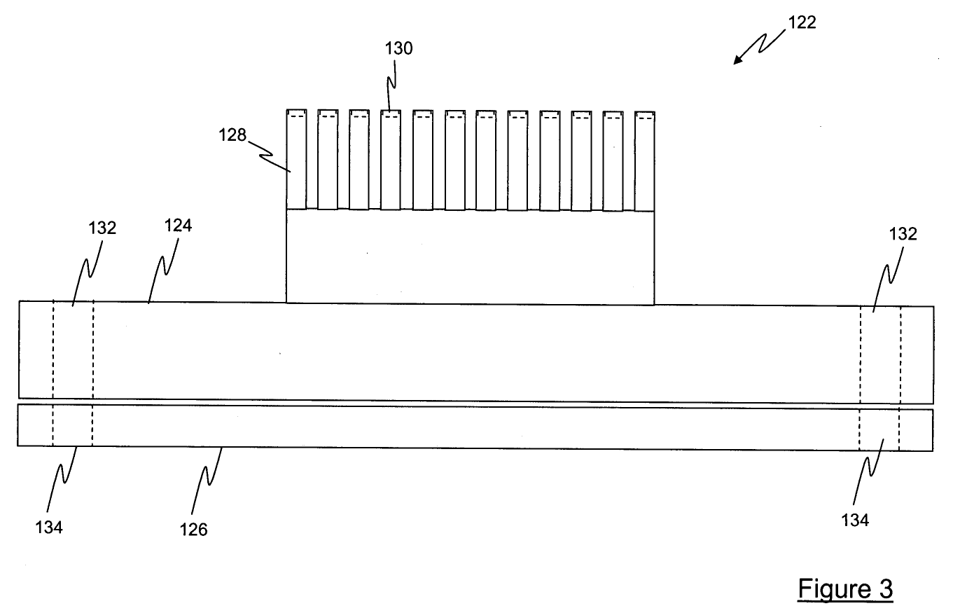

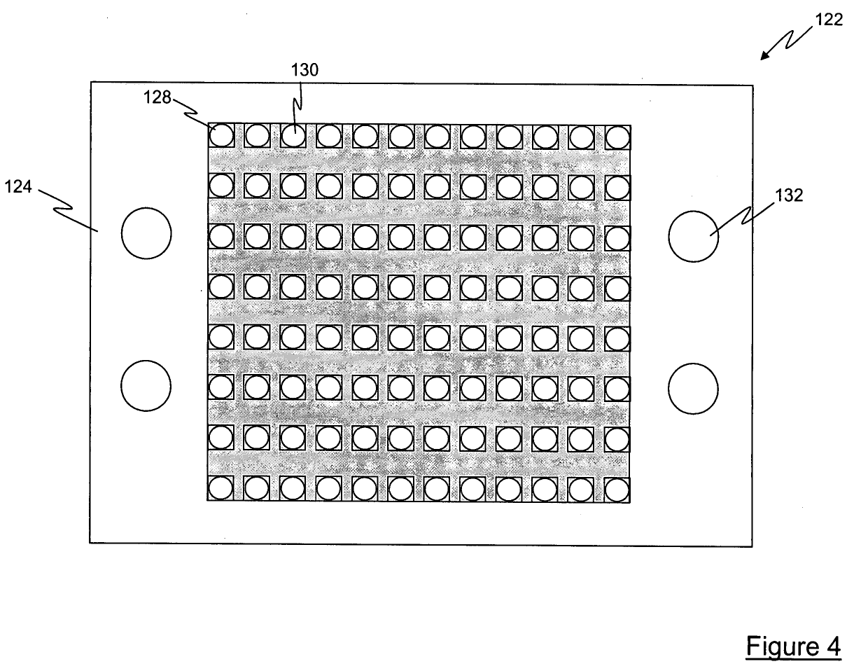

Referring to FIG. 3 and FIG. 4, the micro-tube handling device 100 also includes a tube support device 122 having a tube support device base 124, a tube support device mating plate 126 and a plurality of tube support cradles 128, wherein each of the tube support cradles 128 includes a recess area 130. The tube support device base 124 includes at least one base mounting hole 132 and the tube support device mating plate 126 includes at least one mating plate mounting hole 134, wherein the at least one base mounting hole 132 is aligned with the at least one mating plate mounting hole 134. The tube support device 122 is disposed to be non-movably associated with base actuator interface plate 116 via the tube support mating plate 126, such that when that base actuator 114 is operated, base actuator 114 causes the at least one base actuator piston 120 to move in an upward/downward direction. This in turn causes the base actuator interface plate 116, the tube support device mating plate 126 and the tube support device 122 to move in the direction of motion of the at least one base actuator piston 120. The tube support device base 124, the tube support device mating plate 126 and the base actuator interface plate 116 may be non-movably associated with each other via bolts secured via the at least one mating plate mounting hole 134, the at least one base mounting hole 132 and the at least one base actuator mounting hole 118. However, it should be appreciated that tube support device base 124, tube support device mating plate 126 and the base actuator interface plate 116 may be non-movably associated with each other via method and/or device suitable to the desired end purpose, such as at least one bolt, at least one screw, a frictional connector and/or adhesive.

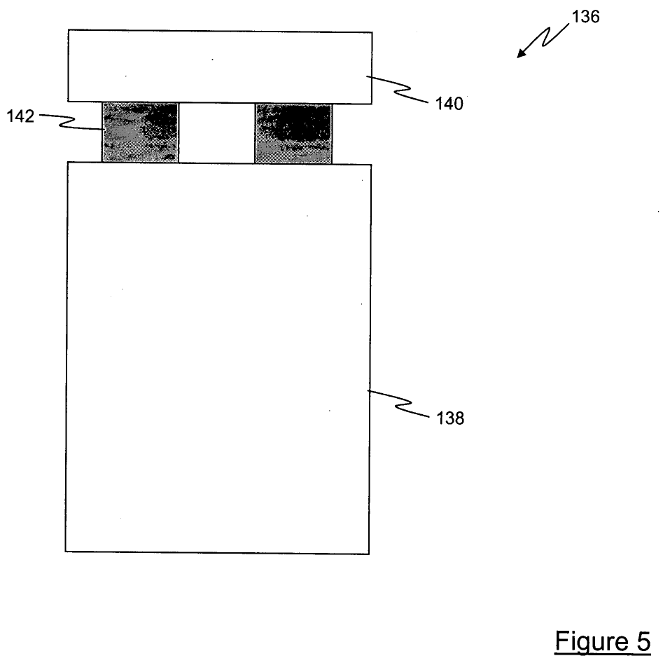

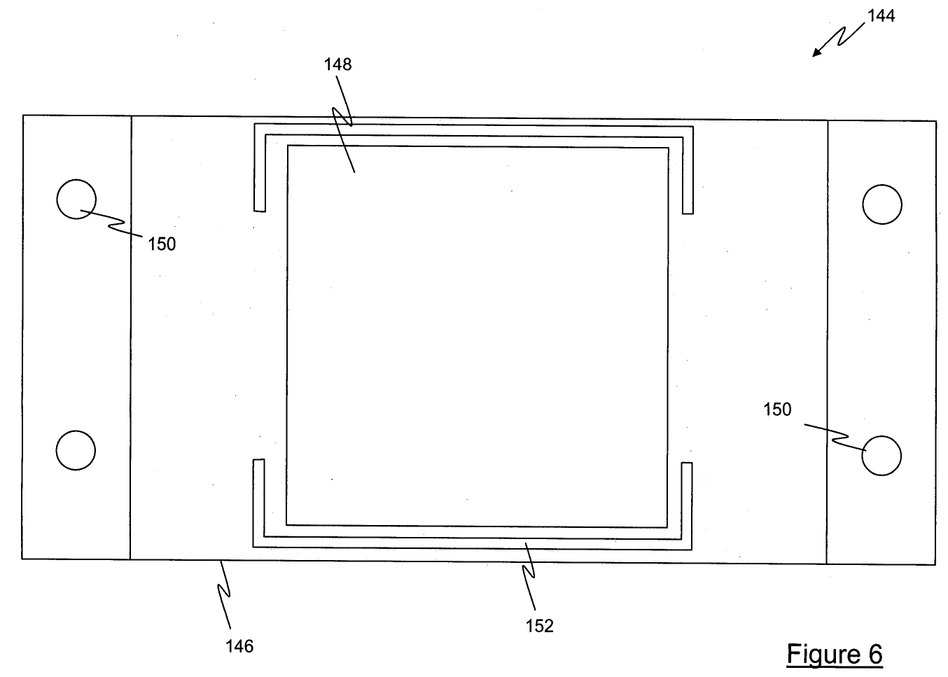

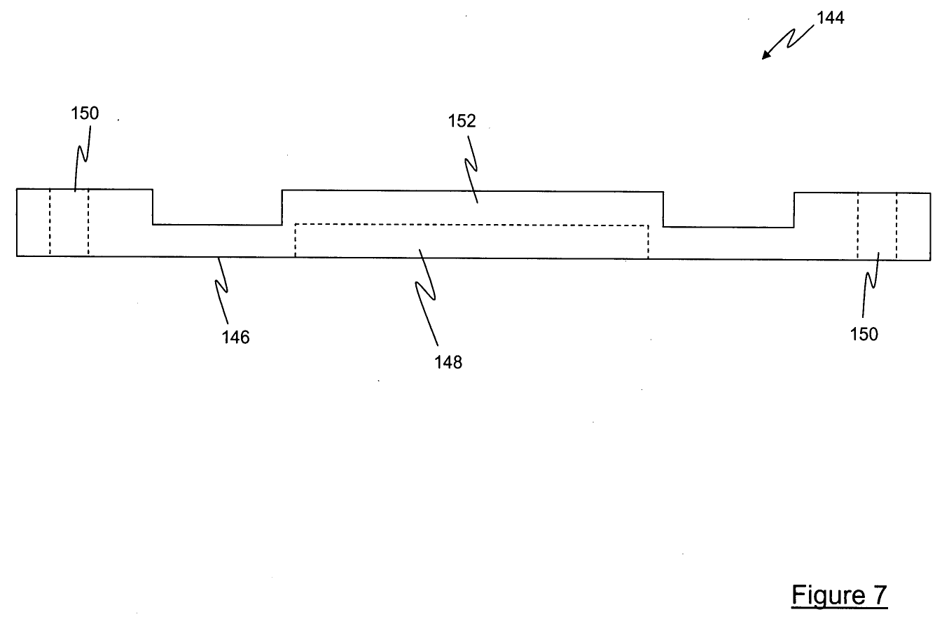

Referring to FIG. 5, the micro-tube handling device 100 also includes a plurality of lower nest actuation devices 136 non-movably disposed on first lower side support wall 106 and second lower side support wall 108. Each of the plurality of lower nest actuation devices 136 includes a lower nest actuator 138 and a lower nest actuator interface plate 140 movably associated with the lower nest actuator 138 via at least one lower nest actuator piston 142. Moreover, each of the lower nest actuator interface plates includes a lower nest plate mounting hole 143. Referring to FIG. 6 and FIG. 7, micro-tube handling device 100 also includes a lower cap-nest 144 having a lower cap-nest structure 146 which defines a lower cap nest opening 148 and at least one lower cap nest mounting hole 150. The lower cap-nest 144 also includes a cradle structure 152 disposed to at least partially surround the lower cap nest opening 148, wherein when a micro-tube rack is disposed within the cradle structure 152, the bottom of the micro-tube rack is accessible via the lower nest opening 148. Referring back to FIG. 1, the lower cap-nest 144 is non-movably associated with the plurality of lower nest actuation devices 136 such that when the lower nest actuation devices 136 is operated, the lower nest actuator 138 causes the at least one lower nest actuator piston 142 to move in an upward/downward direction. This in turn causes the lower nest actuator interface plate 140 and the lower cap-nest 144 to move in the direction of motion of the at least one lower nest actuator piston 142. It should be appreciated that although the lower cap-nest 144 may be non-movably associated with each of the lower nest actuator interface plates 140 using bolts secured via the at least one lower cap nest mounting hole 150 and the lower nest plate mounting hole 143, any method and/or device suitable to the desired end purpose may be used, such as bolts, screws, clips, frictional connector and/or adhesive.

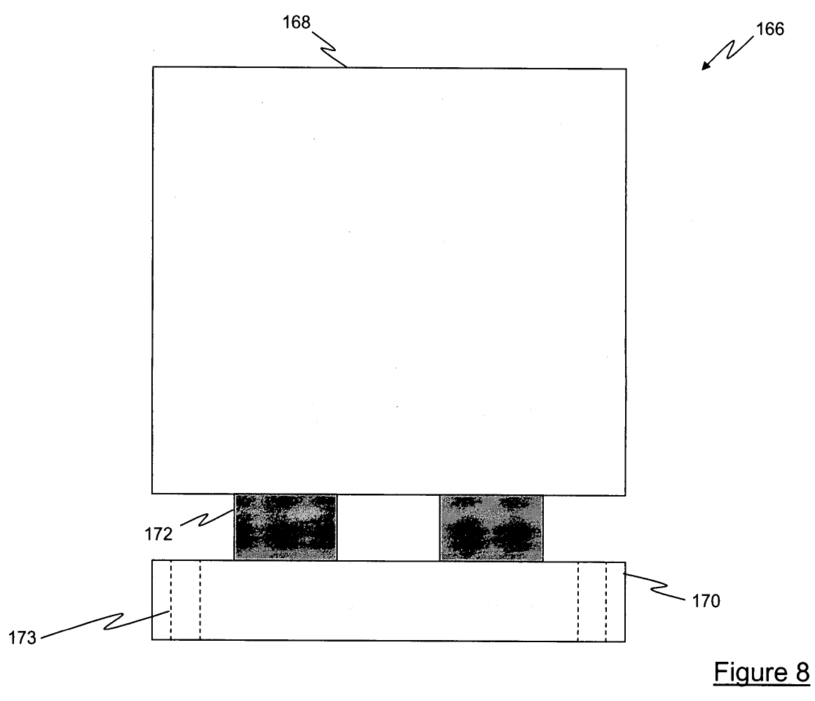

Referring back to FIG. 1, the micro-tube handling device 100 includes an upper support structure 154 having an upper back support wall 156, an upper top surface 158, a first upper side support wall 160 and a second upper side support wall 162, wherein the upper back support wall 156, the upper top surface 158, the first upper side support wall 160 and the second upper side support wall 162 define an upper cavity 164 for containing an upper actuation device 166. As shown in FIG. 8, the upper actuation device 166 includes an upper actuator 168 and an upper actuator interface plate 170, wherein the upper actuator interface plate 170 is movably associated with the upper actuation device 166 via at least one upper actuator piston 172 and includes at least one upper actuator mounting hole 173. Referring back to FIG. 1, it should be appreciated that the upper actuation device 166 is non-movably disposed within the upper cavity 164 such that when the upper actuation device 166 is operated, the upper actuator interface plate 170 extends down and away from the upper top surface 158.

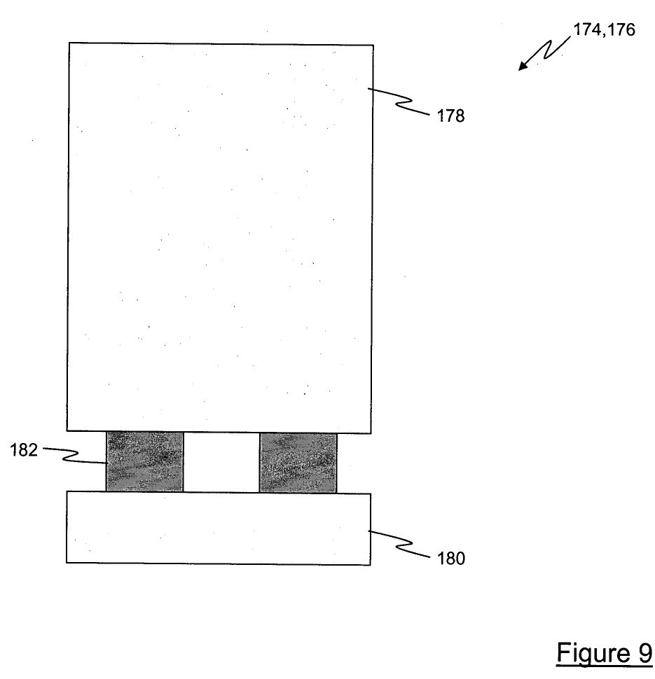

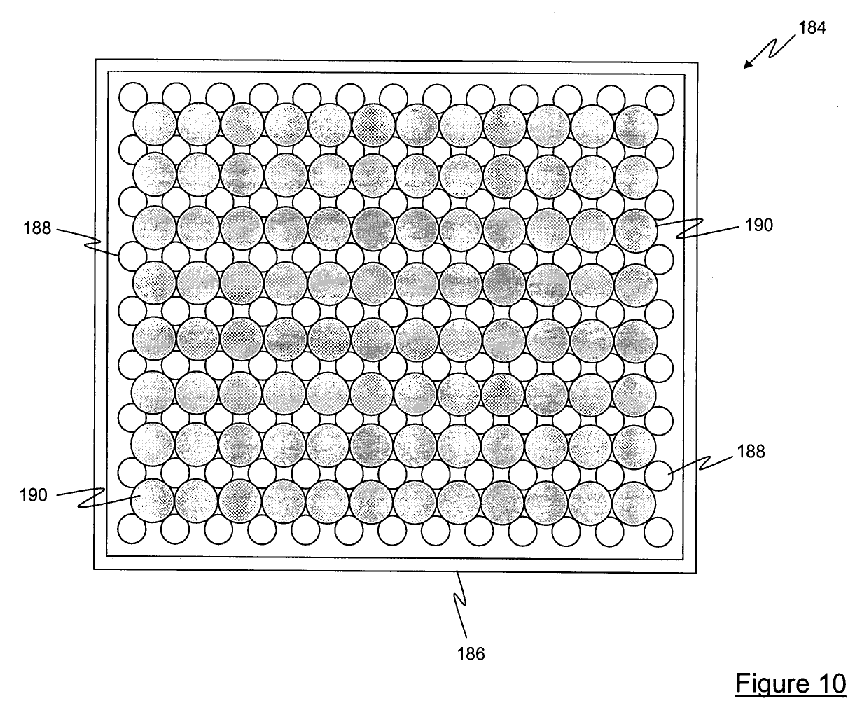

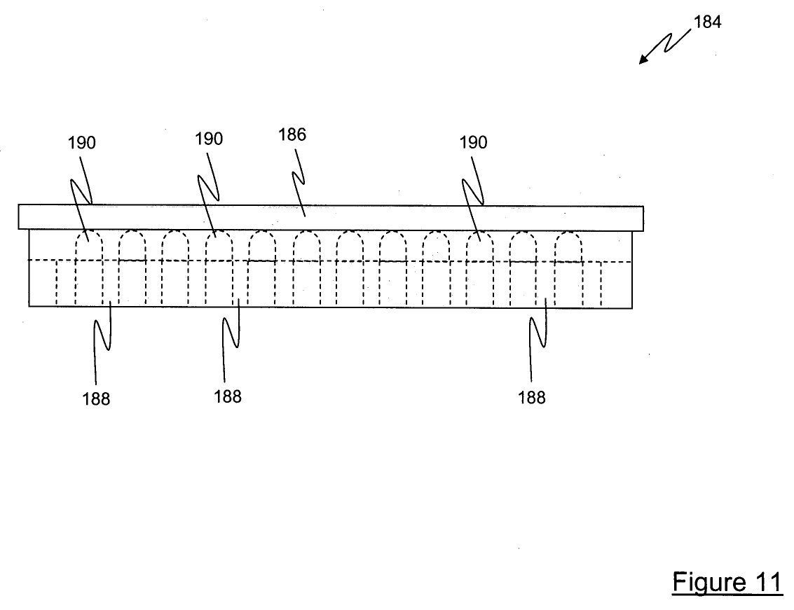

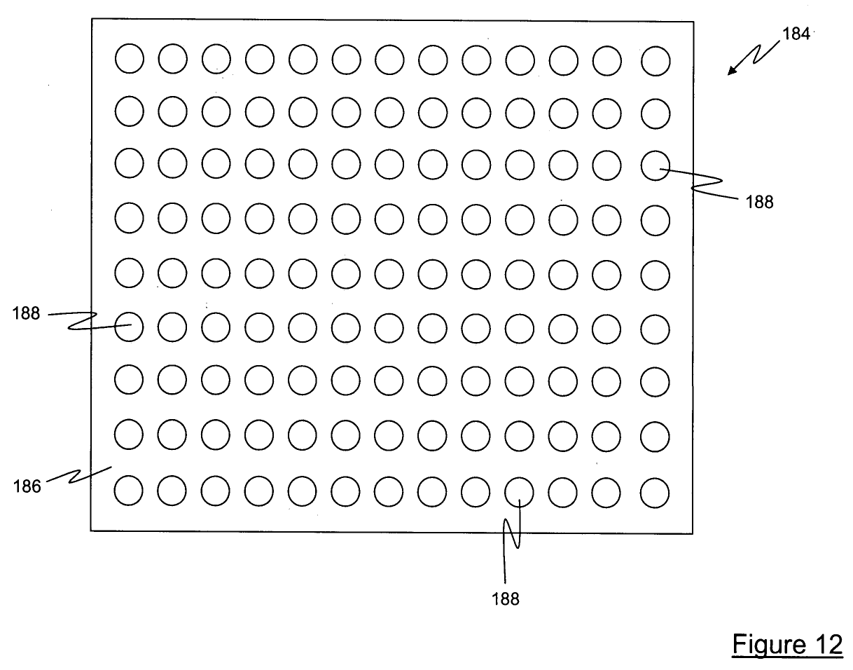

Referring back to FIG. 1, the micro-tube handling device 100 also includes a first upper nest actuation device 174 and a second upper nest actuation device 176, wherein the first upper nest actuation device 174 is non-movably associated with the first upper side support wall 160 and wherein the second upper nest actuation device 176 is associated with the second upper side support wall 162. Referring to FIG. 9, each of the first upper nest actuation device 174 and the second upper nest actuation device 176 includes an upper nest actuator 178 and an upper nest actuator interface plate 180 having at least one upper nest actuator mounting hole 181, wherein the upper nest actuator interface plate 180 is movably associated with the upper nest actuator 178 via at least one upper nest actuator piston 182. It should be appreciated that the first upper nest actuation device 174 is non-movably associated with the first upper side support wall 160 and the second upper nest actuation device 176 is non-movably associated with the second upper side support wall 162, via any securing method and/or device suitable to the desired end purpose, such as bolt, a clip, a screw and/or adhesive. Referring to FIG. 10, FIG. 11 and FIG. 12, the micro-tube handling device 100 further includes a cap-lid device 184 having a cap-lid device structure 186 which defines a plurality of device openings 188 and a plurality of spherical protrusions 190.

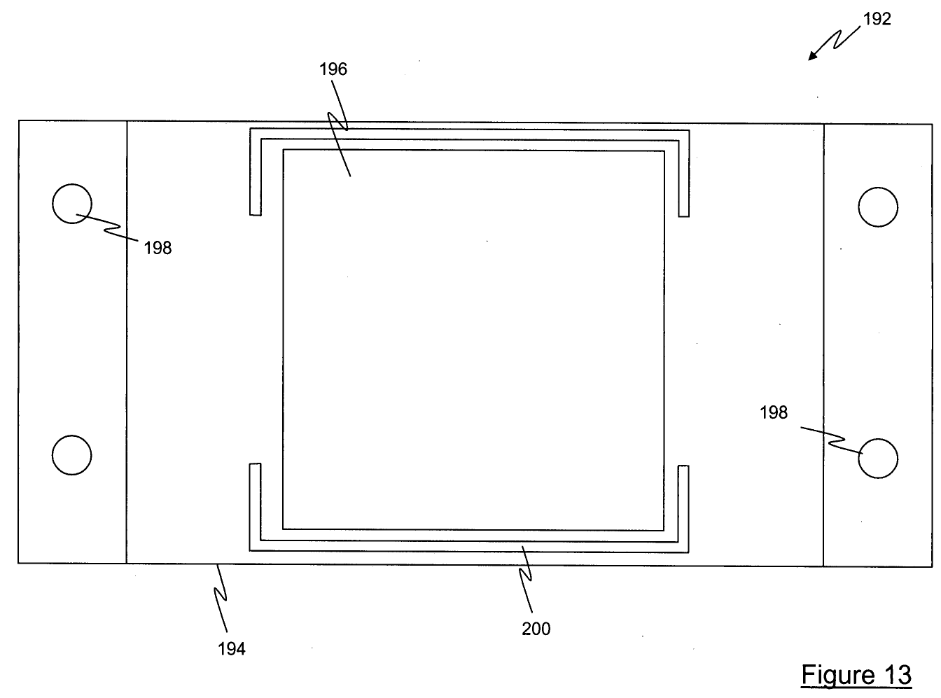

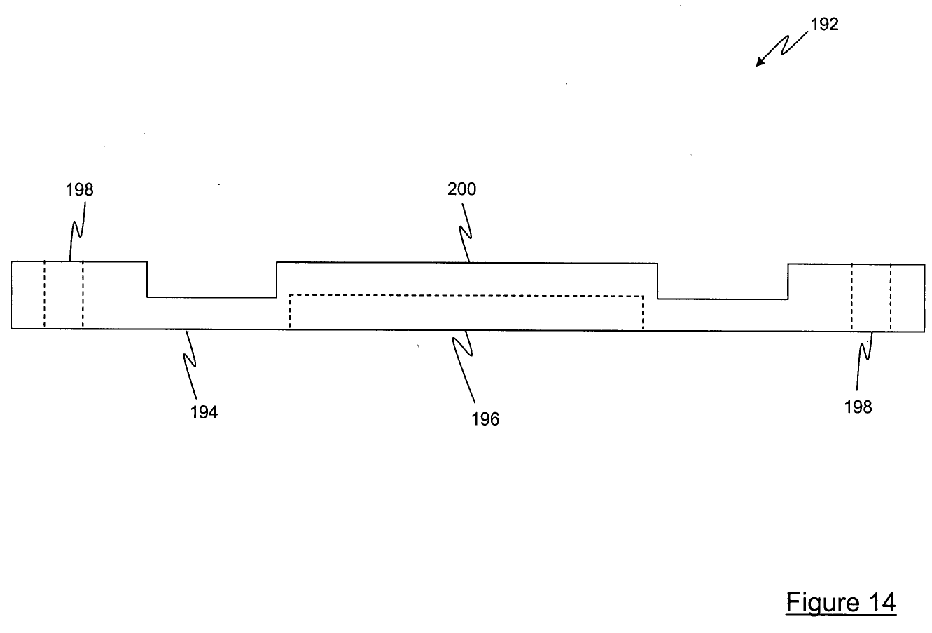

Referring to FIG. 13 and FIG. 14, the micro-tube handling device 100 also includes an upper cap-nest 192, wherein the upper cap-nest 192 includes an upper cap-nest structure 194 which defines an upper cap-nest opening 196 and at least one upper cap-nest mounting hole 198. The upper cap-nest 192 includes a cradle structure 200 disposed to at least partially surround the upper cap-nest opening 196 and is sized to accommodate the cap-lid device 184 which is disposed such that the plurality of spherical protrusions 190 are accessible via the upper cap-nest opening 196. The upper cap-nest 192 may be non-movably associated with the upper nest actuator interface plate 180 by bolting both the first upper nest actuation device 174 and the second upper nest actuation device 176 to the upper nest actuator interface plate 180 of both the first upper nest actuation device 174 and the second upper nest actuation device 176 via the upper nest actuator mounting hole 181 and the upper cap-nest mounting hole 198. However, it should be appreciated that the upper cap-nest 192 may be non-movably associated with the upper nest actuator interface plate 180 via any securing method and/or device suitable to the desired end purpose, such as bolt, a clip, a screw and/or adhesive.

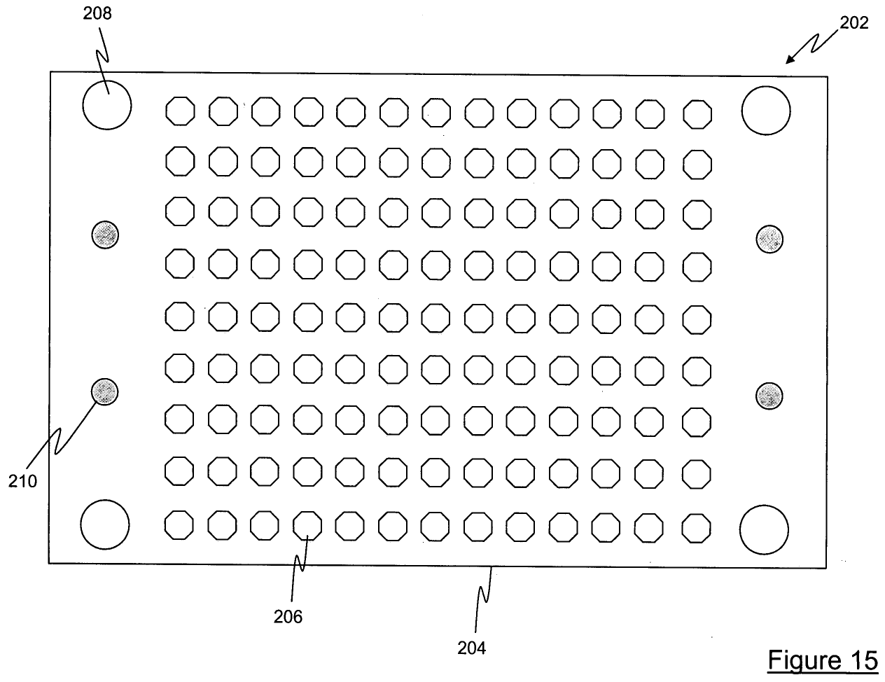

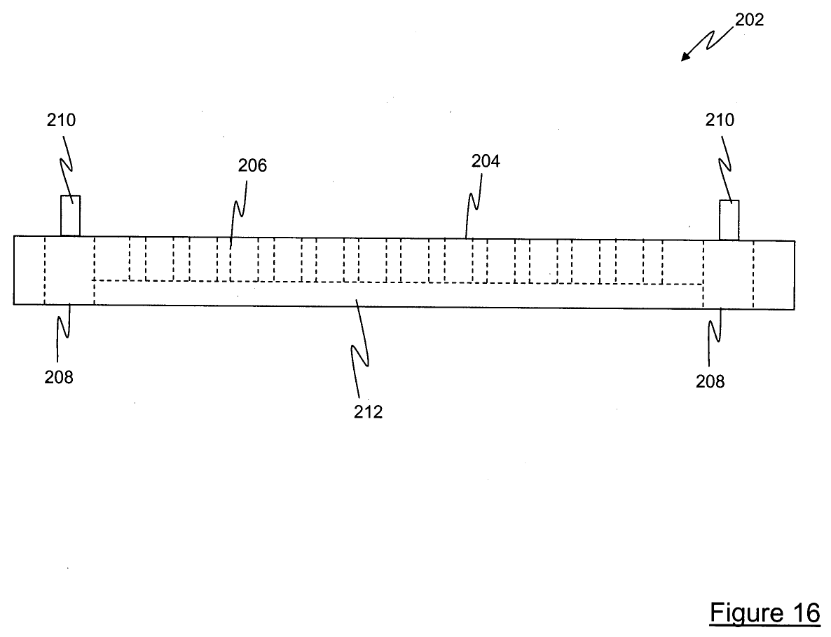

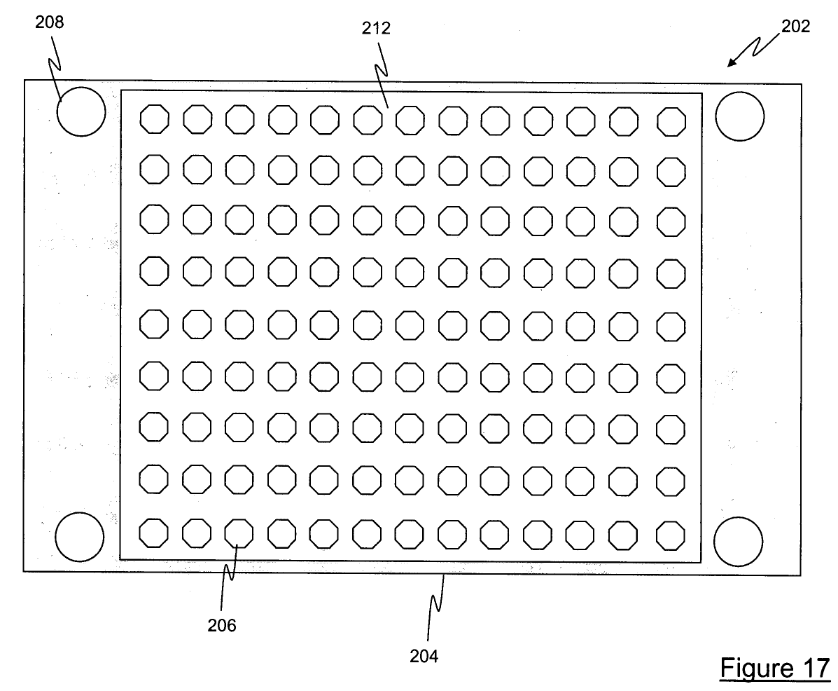

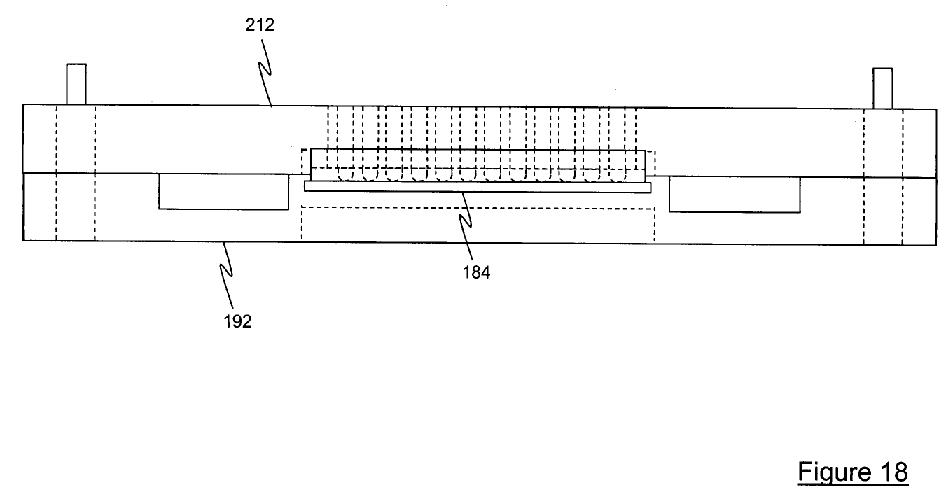

Referring to FIG. 15, FIG. 16 and FIG. 17, the micro-tube handling device 100 further includes a cap-stripper device 202, wherein cap-stripper device 202 includes a cap-stripper device structure 204 which defines a plurality of cap-stripper device openings 206, at least one cap-stripper mounting cavity 208 and at least one cap-stripper mounting post 210. The cap-stripper device structure 204 also defines a cap-stripper device recessed area 212 which is communicated with the plurality of cap-stripper device openings 206. Referring to FIG. 18, the cap-stripper device 202, the upper cap-nest 192 and the cap-lid device 184 are shown associated with each other. As shown, the cap-lid device 184 is associated with the upper cap-nest 192 to be disposed in cradle structure 200 such that the plurality of spherical protrusions 190 are accessible via the upper cap nest opening 196. The cap-stripper device 202 is associated with the upper cap-nest 192 to enclose the cap-lid device 184 between the cap-stripper device 202 and the upper cap-nest 192. The cap-stripper device 202 is non-movably associated with the first upper side support wall 160 and the second upper side support wall 162.

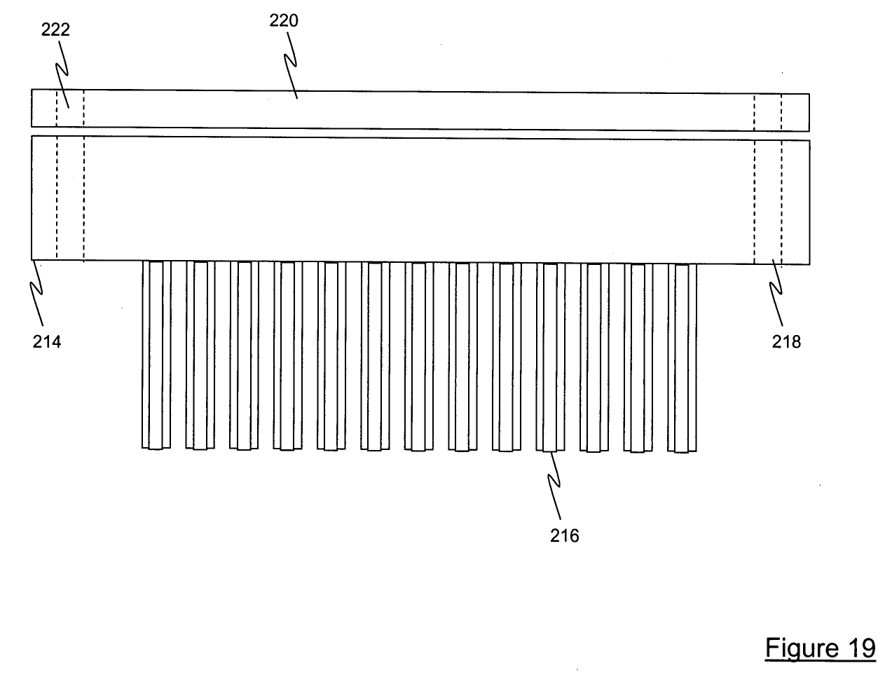

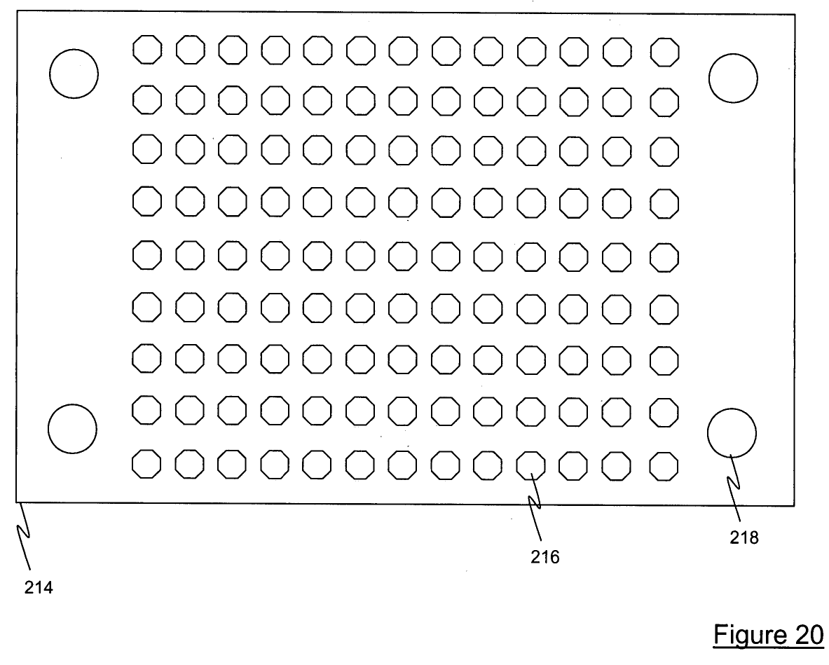

Referring to FIG. 19 and FIG. 20, the micro-tube handling device 100 also includes a stripper device 214 having a plurality of octagonally shaped pins 216 extending therefrom and at least one stripper device mounting hole 218. Additionally, the stripper device 214 is non-movably associated with the upper actuator interface plate 170 via a stripping device mating plate 220 which includes at least one stripping device mating plate mounting hole 222. It should be appreciated that although stripper device 214, stripping device mating plate 220 and upper actuator interface plate 170 are non-movably associated with each other via a bolt connecting stripper device mounting hole 218, stripping device mating plate mounting hole 222 and upper actuator mounting hole 173 together, stripper device 214, stripping device mating plate 220 and upper actuator interface plate 170 may be non-movably associated using any device and or method suitable to the desired end purpose.

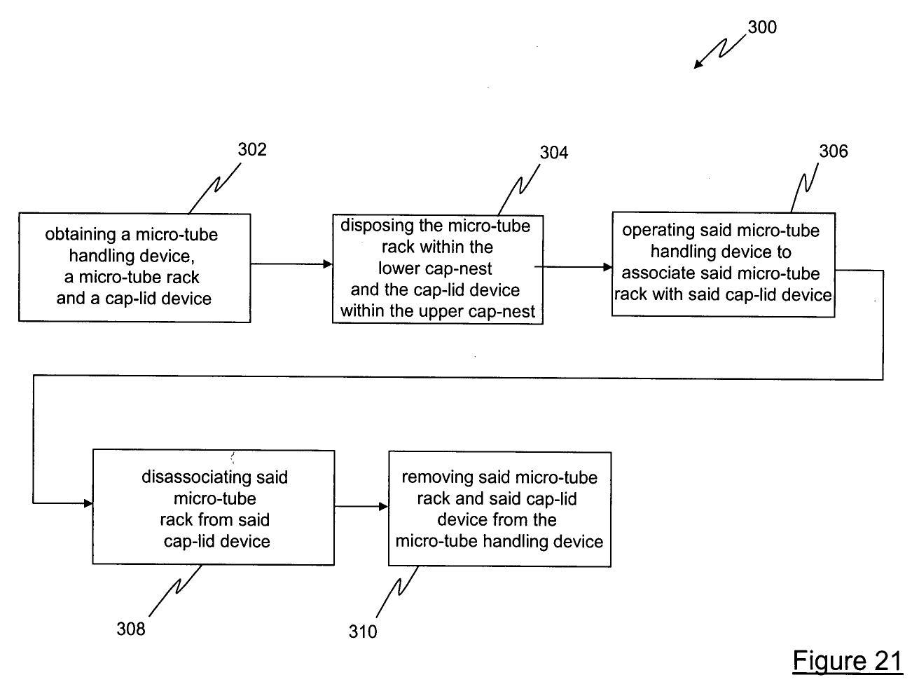

Referring to FIG. 21, a block diagram 300 illustrating a method for implementing the micro-tube handling device 100 is shown and includes obtaining a micro-tube handling device 100, a micro-tube rack 578 and a cap-lid device 184, as shown in operational block 302. The micro-tube handling device 100 includes a lower device portion associated with an upper device portion and at least one micro-tube 580 having a micro-tube opening 586. The cap-lid device 184 includes at least one micro-tube cap 582, wherein the at least one micro-tube cap 582 is frictionally associated with the cap-lid device 184. The micro-tube rack 578 is disposed within the lower device portion and the cap-lid device 184 is disposed with the upper device portion, as shown in operational block 304. This may be accomplished by operating the micro-tube handling device 100 to cause the upper nest actuation device 174,176 to extend the nest actuator piston 182, and thus upper nest actuator interface plate 180, away from cap-stripper device 202. This exposes the cradle structure 200 and the upper cap-nest opening 196. The cap-lid device 184 is then disposed within the cradle structure 200 such that the plurality of spherical protrusions 190 are adjacent the upper cap-nest opening 196. The micro-tube handling device 100 is then operated to cause the nest actuator interface plate 180 to contract toward the upper nest actuation device 174,176. This brings the cap-lid device 184 into coincidence with the cap-stripper device 202. In a similar fashion, the micro-tube rack is disposed within the lower cap-nest 144 such that the micro-tube plurality of micro-tube rack is between the micro-tubes and the lower cap-nest 144. It should be appreciated that this may be accomplished using manual means, such as by a technician, and/or by automatic means, such as by a robotic device.

The micro-tube handling device 100 may then be operated to associate the micro-tube rack 578 with the cap-lid device 184 such that the micro-tube cap 582 is snugly contained within the micro-tube opening 586, as shown in operational block 306. This may be accomplished by operating the micro-tube handling device 100 to raise the lower cap-nest 144, and hence the micro-tube rack 578, toward the cap-lid device 184 such that the micro-tube caps 582 are snugly disposed within the micro-tubes on the micro-tube rack 578. The micro-tube rack 578 may then be disassociated from the cap-lid device 184 such that the micro-tube cap 582 is disassociated from the cap-lid device 184, as shown in operational block 308. This may be accomplished by lowering the stripper device 214 such that the plurality of octagonally shaped pins 216 cause the micro-tubes and the micro-tube caps 582 to become disassociated from the cap-lid device 184. The micro-tube rack 578 and the cap-lid device 184 may then be removed from the micro-tube handling device 100 and replaced as discussed hereinabove, as shown in operational block 310.

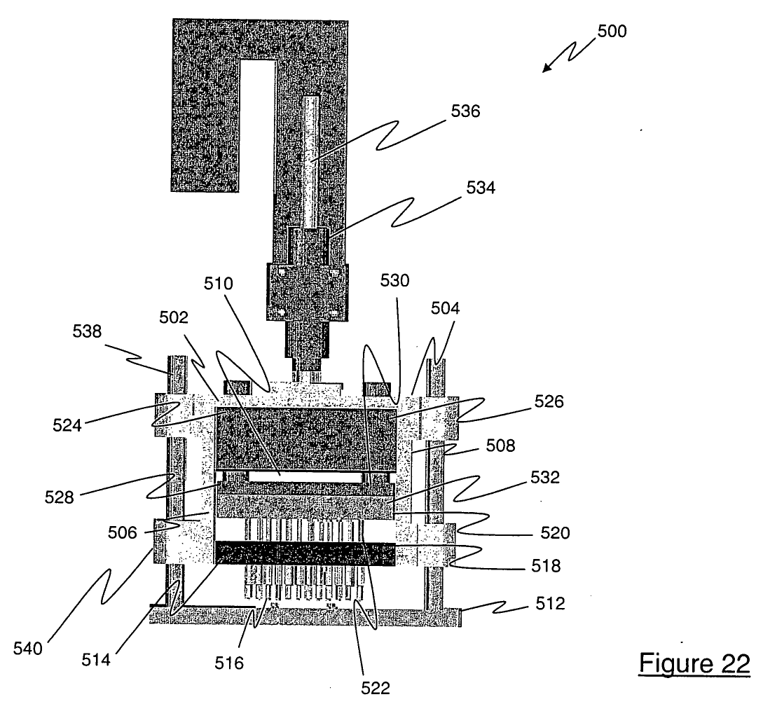

Referring to FIG. 22, a second embodiment of a micro-tube handling device 500 is shown and includes a micro-tube handling device support structure 502 having a structure top 504, a first side structure 506 and a second side structure 508, wherein the structure top 504, the first side structure 506 and the second side structure 508 define a structure cavity 510. The micro-tube handling device 500 also includes a stripper plate 512 having a plurality of stripper plate holes 513, a collet holder 514 for holding a plurality of collets 516, a collet retainer 518 and a pin holder 520 for holding a plurality of collet pins 522. An actuation device 524 is also provided and includes a device actuator 526 and a device actuator mating plate 528, wherein device actuator mating plate 528 is movably associated with the device actuator 526 via at least one device actuator piston 530 which controllably extends from and contracts into device actuator 526.

The actuation device 524 is non-movably disposed within the structure cavity 510 such that the actuation device 524 is adjacent the structure top 504 and such that the device actuator mating plate 528 is disposed away from the top structure 504. The pin holder 520 is non-movably associated with the device actuator mating plate 528 via a collet pin mating plate 532 such that operating the device actuator 526 to cause the device actuator mating plate 528 to controllably extend from the device actuator 526 also causes the collet pin mating plate 530 to move away from the device actuator 526. The micro-tube handling device 500 further includes an upper actuator 534 which is movably associated with the structure top 504 via at least one upper actuator piston 536. Furthermore, the micro-tube handling device 500 further includes at least one main piston 538 non-movably associated with the stripper plate 512 and at least one micro-tube handling device bearing 540 perpendicularly protruding from the first side structure 506 and the second side structure 508. It should be appreciated that the main piston 538 is movably disposed within the micro-tube handling device bearing 540, to allow the collet holder 514, the pin holder 520 and the actuation device 524 to move up and down relative to the stripper plate 512.

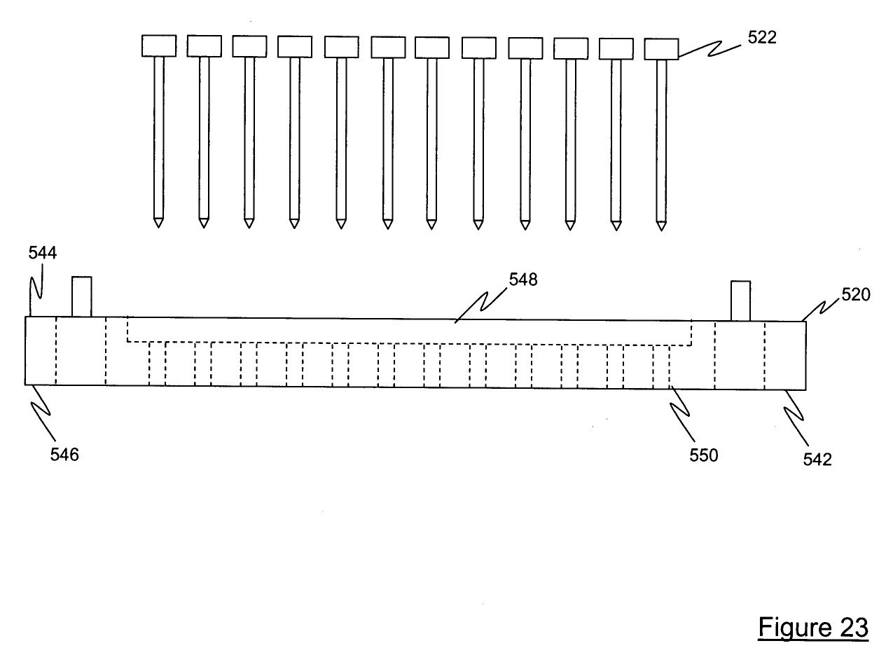

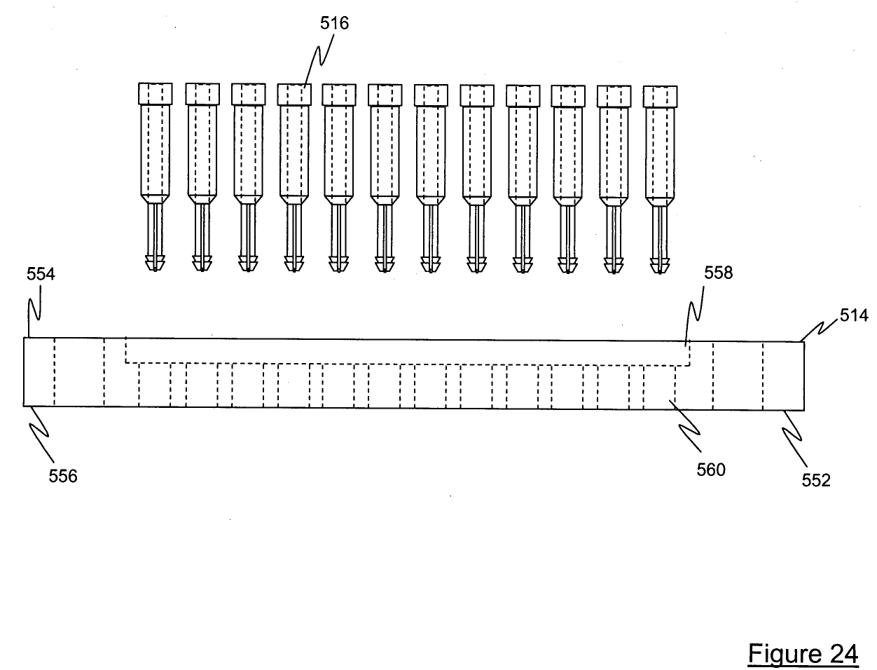

Referring to FIG. 23, the pin holder 520 includes a collet pin holder plate structure 542 having a collet pin holder plate top 544 and a collet pin holder plate bottom 546, wherein the collet pin holder plate top 544 defines a collet pin holder plate recessed portion 548 having a plurality of collet pin holes 550 for holding the plurality of collet pins 522, wherein the collet pin holes 550 traverse the width of the collet pin holder 520 such that the collet pin holder plate top 544 is communicated with the collet pin holder plate bottom 546. It should be appreciated that although the collet pin holder plate recessed portion 548 is shown as having ninety-six collet pin holes 550, the collet pin holder plate recessed portion 548 may have any number of collet pin holes 550 suitable to the desired end purpose. Referring to FIG. 24, the collet holder 514 includes a collet holding plate structure 552 having a collet holding plate structure top 554 and a collet holding plate structure bottom 556, wherein the collet holding plate structure top 554 defines a collet holding plate recessed portion 558 having a plurality of collet holes 560 for holding the plurality of collets 516, wherein the plurality of collet holes 560 traverse the width of the collet holder 514 such that the collet holding plate structure top 554 is communicated with the collet holding plate structure bottom 556. It should be appreciated that although the collet holding plate recessed portion 558 is shown as having ninety-six collet holes 560, the collet holding plate recessed portion 558 may have any number of collet holes 560 suitable to the desired end purpose.

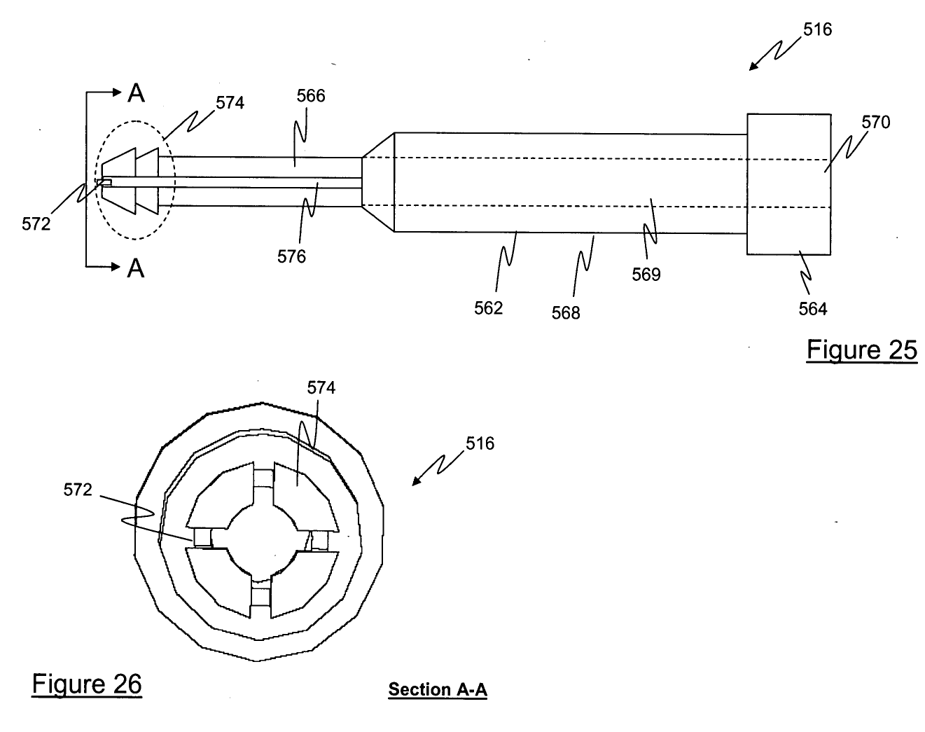

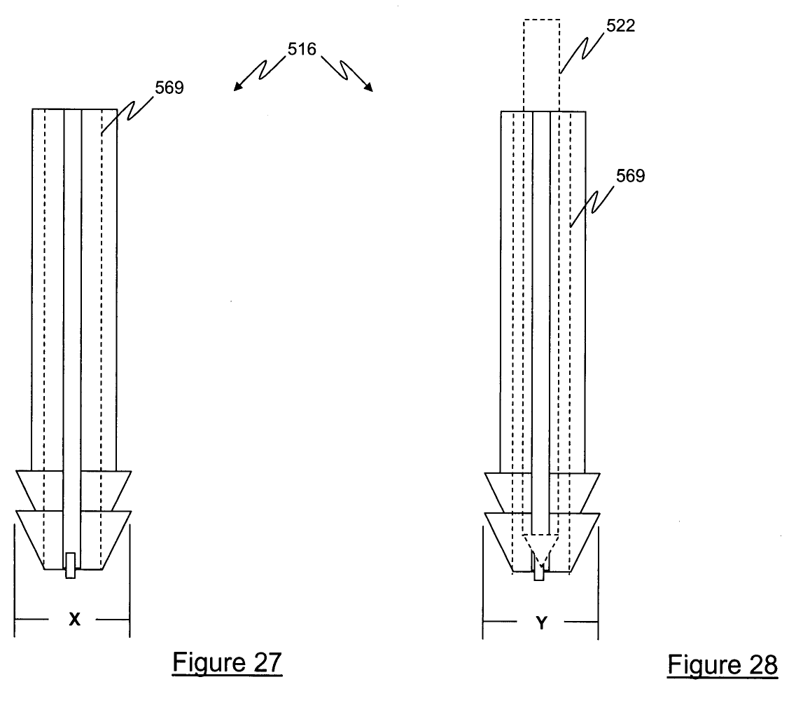

Referring to FIG. 25 and FIG. 26, one of the plurality of collets 516 is shown and includes a collet structure 562 having a collet head portion 564 associated with a collet tip portion 566 via a collet body portion 568. The collet structure 562 defines a pin cavity 569 having a pin cavity inlet 570 disposed in collet head portion 564 and a pin cavity outlet 572 disposed in collet tip portion 566, wherein the pin cavity 564 traverses the entire length of the collet 516 to communicate the collet head portion 564 with the collet tip portion 566. The collet tip portion 566 includes at least one collet tip barbed portion 574 and defines a plurality of collet tip channels 576 which traverse the length of collet tip portion 566. This allows the width of collet tip portion 566 to be increased in a controlled manner between a first collet width X and a second collet width Y. Referring to FIG. 27, a collet 516 is shown with the pin cavity 569 empty and having a first collet width X. When a collet pin 522 is disposed within the pin cavity 569, as shown in FIG. 28, the collet pin 522 causes the width of the collet tip portion 566 to change from the first collet width X to the second collet width Y, wherein the second collet width Y is larger than the first collet width X.

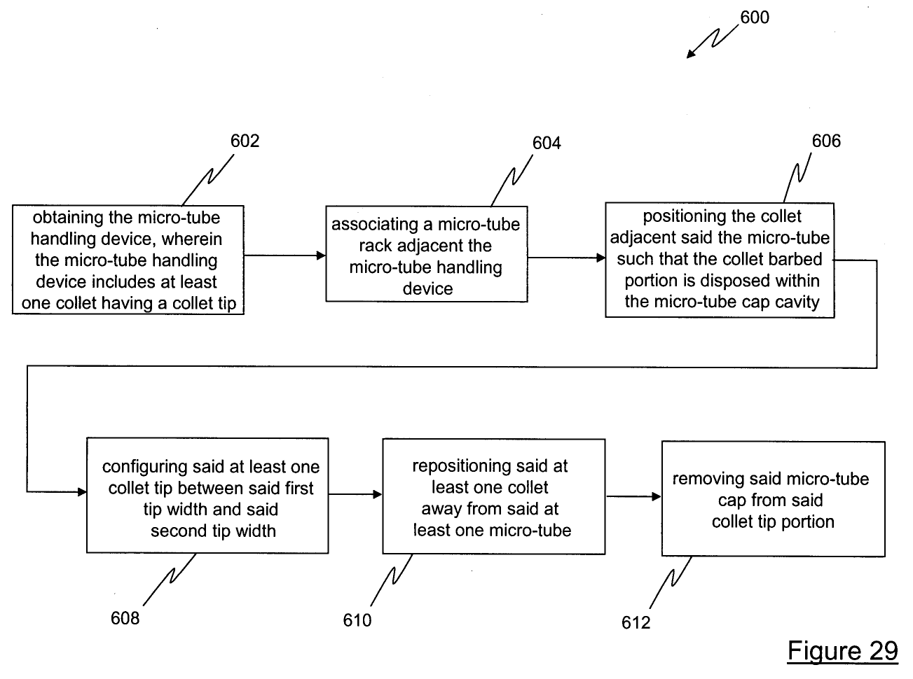

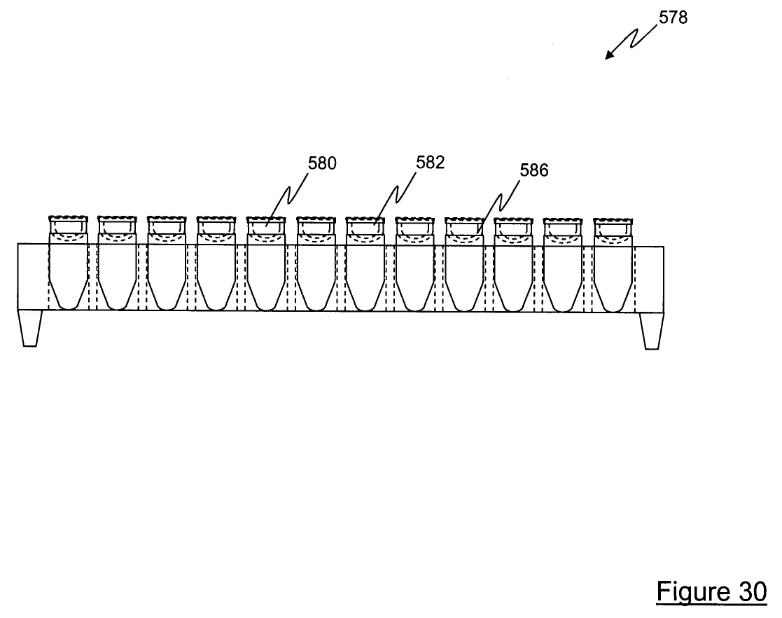

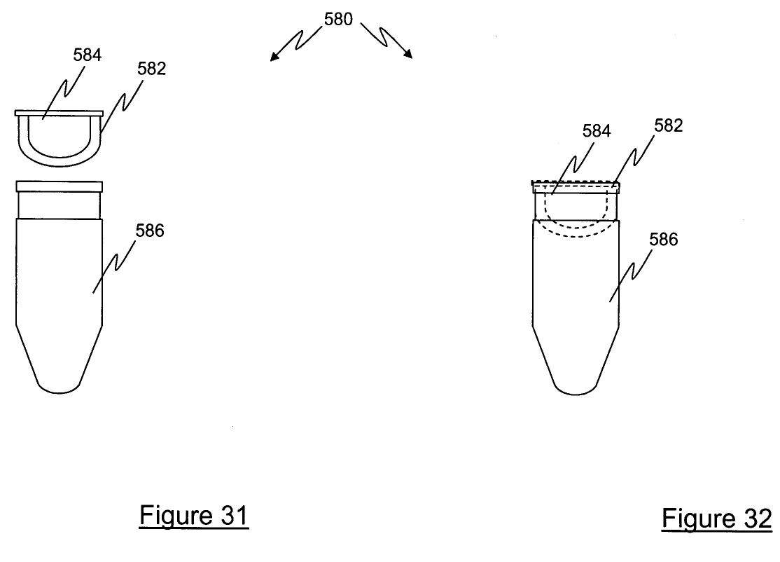

Referring to FIG. 29, a block diagram 600 illustrating a method for implementing the micro-tube handling device 500 is shown and includes obtaining the micro-tube handling device 500, wherein the micro-tube handling device includes at least one collet 516 having a collet tip portion 566, as shown in operational block 602. The collet tip portion 566 includes a collet tip barbed portion 568 and is sizably configurable between a first collet width X and a second collet width Y. A micro-tube rack 578, as shown in FIG. 30, is disposed adjacent the micro-tube handling device 500 to associate the micro-tube rack 578 with the micro-tube handling device 500, as shown in operational block 604. This may be accomplished by positioning the micro-tube rack 578 under the stripper plate 512 such that the micro-tubes 580 in the micro-tube rack 578 are aligned with the plurality of stripper plate holes 513. Referring to FIG. 31 and FIG. 32, it should be appreciated that the micro-tube rack 578 includes at least one micro-tube 580 which defines a micro-tube cavity 581 and having a micro-tube cap 582 which defines a cap cavity 584.

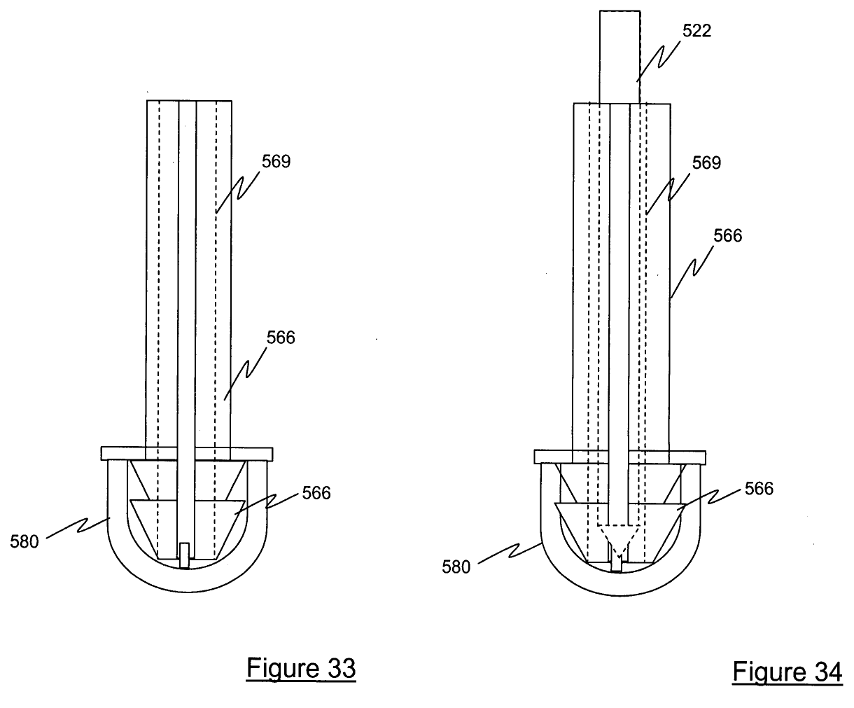

The at least one collet 516 may then be positioned to be associated with the at least one micro-tube 580, as shown in operational block 606, such that the collet tip barbed portion 568 is at least partially disposed within the cap cavity 584, as shown in FIG. 33. This may be accomplished by operating the micro-tube handling device 500 to cause the upper actuator 534 to move the device support structure 502 toward stripper plate 512, such that the collet tip portion 566, and hence the collet tip barbed portion 574, of the plurality of collets 516 traverses the stripper plate holes 513 to be disposed within the cap cavity 584. The collet tip portion 566 may then be configured between the first collet width X and the second collet width Y to cause the collet tip barbed portion 568 to securingly interact with the micro-tube cap 580, as shown in FIG. 34 and operational block 608. This may be accomplished by lowering the collet pin holder 520 such that the plurality of collet pins 522 are disposed within the pin cavity 564 of the collets 516 to engage the collet tip portion 566 causing the collet tip portion 566 to spread thus increasing the width of the collet tip portion 566 to be configured between the first collet width X and the second collet width Y. This, in turn, causes the collet tip barbed portion 568 to be grippingly compressed into the micro-tube walls forming cap cavity 584. The at least one collet 516 may then be repositioned away from the at least one micro-tube 580, as shown in operational block 610, causing the micro-tube cap 582 to be removed from the micro-tube cavity 581. The micro-tube cap 582 is then removed from the collet tip portion 566, as shown in operational block 612. This may be accomplished by raising the collet pin holder 520 such that the plurality of collet pins 522 are removed from the pin cavity 564 of the collets 516 to cause the collet tip portion 566 to be configured between the second collet width Y and the first collet width X. As such, the collet tip barbed portion 568 is no longer grippingly compressed into the micro-tube walls forming the cap cavity 584 and the micro-tube cap 582 is allowed to fall away from the collet tip portion 566, where the removed micro-tube caps 582 may be vacuumed into a receptacle for further use/reuse or for discard.

It should be appreciated that the upper actuation device 166, the base actuation device 112, the upper nest actuation device 174, 176 and the lower nest actuation device 136 is at least one of a pneumatic actuation device, an electric actuation device and an electro-pneumatic actuation device. It should be further appreciated that the micro-tube handling device may be constructed, in whole or in part, from any material or combination of materials suitable to the desired end purpose, such as a metallic material, a ceramic material, a plastic material and a composite material.

It should be appreciated that the method of FIG. 21 and FIG. 29 may be implemented through a processing device operating in response to a computer program. In order to perform the prescribed functions and desired processing, as well as the computations therefore (e.g., the execution of fourier analysis algorithm(s), the control processes prescribed herein, and the like), the controller may include, but not be limited to, a processor(s), computer(s), memory, storage, register(s), timing, interrupt(s), communication interfaces, and input/output signal interfaces, as well as combinations comprising at least one of the foregoing. For example, the controller may include signal input signal filtering to enable accurate sampling and conversion or acquisitions of such signals from communications interfaces. It is also considered within the scope of the invention that the processing of FIG. 24 may be implemented by a controller located remotely from the processing device.

It should also be appreciated that the method of FIG. 21 and FIG. 29 may be embodied in the form of computer-implemented processes and apparatuses for practicing those processes. The above can also be embodied in the form of computer program code containing instructions embodied in tangible media, such as floppy diskettes, CD-ROMs, hard drives, or any other computer-readable storage medium, wherein, when the computer program code is loaded into and executed by a computer, the computer becomes an apparatus for practicing the invention. Existing systems having reprogrammable storage (e.g., flash memory) can be updated to implement the invention. The above can also be embodied in the form of computer program code, for example, whether stored in a storage medium, loaded into and/or executed by a computer, or transmitted over some transmission medium, such as over electrical wiring or cabling, through fiber optics, or via electromagnetic radiation, wherein, when the computer program code is loaded into and executed by a computer, the computer becomes an apparatus for practicing the invention. When implemented on a general-purpose microprocessor, the computer program code segments configure the microprocessor to create specific logic circuits.

While the invention has been described with reference to an exemplary embodiment, it will be understood by those skilled in the art that various changes, omissions and/or additions may be made and equivalents may be substituted for elements thereof without departing from the spirit and scope of the invention. In addition, many modifications may be made to adapt a particular situation or material to the teachings of the invention without departing from the scope thereof. Therefore, it is intended that the invention not be limited to the particular embodiment disclosed as the best mode contemplated for carrying out this invention, but that the invention will include all embodiments falling within the scope of the appended claims. Moreover, unless specifically stated any use of the terms first, second, etc. do not denote any order or importance, but rather the terms first, second, etc. are used to distinguish one element from another.

We claim:

1. A micro-tube handling device, comprising:
a lower cap-nest, wherein said lower cap-nest includes a rack cradle surrounding a rack nest opening;
a tube support device, wherein said tube support device is disposed to be associated with said rack nest opening to allow said tube support device access to said lower cap-nest via said rack nest opening;
a cap-lid device, wherein said cap-lid device includes a cap retainer portion disposed adjacent a plurality of a cap-lid cavities; and
a stripper block device, wherein said stripper block device includes a plurality of stripper block pins, wherein each of said plurality of stripper block pins are disposed to be aligned with each of said plurality of cap-lid cavities.

2. The micro-tube handling device of claim 1, wherein said lower cap-nest is movably associated with the micro-tube handling device via at least one lower nest actuation device.

3. The micro-tube handling device of claim 1, wherein said tube support device includes a plurality of tube support cradles, wherein each of said plurality of tube support cradles includes a recessed area.

4. The micro-tube handling device of claim 1, wherein said tube support device is movably associated with the micro-tube handling device via a base actuation device.

5. The micro-tube handling device of claim 1, wherein said cap-lid device includes at least one micro-tube cap removably associated with said cap retainer portion.

6. The micro-tube handling device of claim 1, wherein said cap retainer portion includes a plurality of spherical protrusions.

7. The micro-tube handling device of claim 1, further comprising an upper cap-nest device movably associated with the micro-tube handling device via at least one upper nest actuation device.

8. The micro-tube handling device of claim 1, wherein said plurality of stripper block pins are sized and shaped to traverse said plurality of cap-lid cavities.

9. The micro-tube handling device of claim 1, wherein said stripper block pins are octagonal in shape to allow said stripper block pins to be positioned adjacent said cap retainer portion, wherein said cap retainer portion includes a plurality of spherical protrusions.

10. The micro-tube handling device of claim 1, wherein said stripper block device is movably associated with the micro-tube handling device via an upper actuation device.

11. The micro-tube handling device of claim 1, wherein said upper actuation device, said base actuation device, said upper nest actuation device and said lower nest actuation device is at least one of a pneumatic actuation device, an electric actuation device and an electro-pneumatic actuation device.

12. The micro-tube handling device of claim 1, wherein the micro-tube handling device is constructed from at least one of a metallic material, a ceramic material, a plastic material and a composite material.

13. A method for implementing a micro-tube handling device, the method comprising:
obtaining a micro-tube handling device, a micro-tube rack and a cap-lid device, wherein said micro-tube handling device includes a lower cap-nest movably associated with an upper cap-nest, and wherein said micro-tube rack includes at least one micro-tube having a micro-tube opening and wherein said cap-lid device includes at least one micro-tube cap, wherein said at least one micro-tube cap is frictionally associated with said cap-lid device;
disposing said micro-tube rack within said lower cap-nest and said cap-lid device within said upper cap-nest;
operating said micro-tube handling device to associate said micro-tube rack with said cap-lid device such that said at least one micro-tube cap is snugly contained within said micro-tube opening;
disassociating said micro-tube rack from said cap-lid device such that said at least one micro-tube cap is disassociated from said cap-lid device;
removing said micro-tube rack and said cap-lid device from the micro-tube handling device.

14. The method of claim 13, wherein said disposing includes lowering said upper cap nest to allow access to a cradle structure disposed on said upper cap nest.

15. The method of claim 13, wherein said cap-lid device includes at least one micro-tube cap retaining portion and wherein said disposing includes disposing said cap-lid device such that said micro-tube cap retaining portion is adjacent said upper cap-nest.

16. The method of claim 13, wherein said disposing includes disposing said micro-tube rack such that said plurality of micro-tubes are separated from said lower cap-nest via said micro-tube rack.

17. The method of claim 13, wherein said operating includes raising said lower cap-nest such that said at least one micro-tube cap is frictionally associated with said at least one micro-tube.

18. The method of claim 13, wherein said micro-tube handling device further includes a stripper device having a plurality of octagonally shaped pins and wherein said disassociating includes positioning said stripper device such that said plurality of octagonally shaped pins compressingly contact said cap-lid device to separate said at least one micro-tube cap from said cap-lid device.

19. A machine-readable computer program code, the program code including instructions for causing a controller to implement method for implementing a micro-tube handling device, wherein the micro-tube handling device includes a micro-tube rack, a cap-lid device and a lower device portion movably associated with an upper device portion and wherein the micro-tube rack includes at least one micro-tube having a micro-tube opening and wherein the cap-lid device includes at least one micro-tube cap, wherein the at least one micro-tube cap is frictionally associated with the cap-lid device, the method comprising:
disposing the micro-tube rack within the lower device portion and the cap-lid device within the upper device portion;
operating the micro-tube handling device to associate the micro-tube rack with the cap-lid device such that the at least one micro-tube cap is snugly contained within the micro-tube opening;
disassociating the micro-tube rack from the cap-lid device such that the at least one micro-tube cap is disassociated from the cap-lid device;
removing the micro-tube rack and the cap-lid device from the micro-tube handling device.

20. The machine-readable computer program code of claim 19, wherein the machine-readable computer program code is encoded onto a storage medium.

21. A micro-tube handling device, comprising:
at least one collet pin; and
at least one collet, wherein said at least one collet includes a sizably configurable collet tip having at least one barbed portion and wherein said at least one collet defines a collet pin cavity for receiving said at least one collet pin, wherein said sizably configurable collet tip is configurable between a first collet tip width and a second collet tip width by disposing said at least one collet pin within said collet pin cavity.

22. The micro-tube handling device of claim 21, wherein said at least one collet pin includes a plurality of collet tip channels associated with said collet tip.

23. The micro-tube handling device of claim 21, wherein said at least one barbed portion includes two barbed portions.

24. The micro-tube handling device of claim 21, wherein said collet pin is sized and shaped such that when said collet pin is disposed within said collet pin cavity, said collet tip is configured from said first collet tip width to said second collet tip width.

25. The micro-tube handling device of claim 24, wherein said second collet tip width is larger than said first collet tip width.

26. The micro-tube handling device of claim 21, further comprising an upper actuator and an actuation device.

27. The micro-tube handling device of claim 26, wherein said actuation device and said upper actuator is at least one of a pneumatic actuation device, an electric actuation device and an electro-pneumatic actuation device.

28. The micro-tube handling device of claim 21, wherein the micro-tube handling device is constructed from at least one of a metallic material, a ceramic material, a plastic material and a composite material.

29. The micro-tube handling device of claim 21, further including at least one main piston and at least one micro-tube handling device bearing, wherein said at least one main piston is movably disposed within said at least one micro-tube handling device bearing.

30. A method for implementing a micro-tube handling device, the method comprising:
obtaining the micro-tube handling device, wherein the micro-tube handling device includes at least one collet having a collet tip, wherein said collet tip includes a collet barbed portion and is sizably configurable between a first tip size and a second tip size;
associating a micro-tube rack adjacent the micro-tube handling device, wherein said micro-tube rack includes at least one micro-tube having a micro-tube cap which defines a cap cavity;
positioning said at least one collet adjacent said at least one micro-tube such that said collet barbed portion is at least partially disposed within said micro-tube cap cavity;
configuring said at least one collet tip between said first tip width and said second tip width to cause said collet barbed portion to securingly interact with said micro-tube cap;
repositioning said at least one collet away from said at least one micro-tube to cause said micro-tube cap to be removed from said micro-tube cavity; and
removing said micro-tube cap from said collet tip portion.

31. The method of claim 30, wherein said associating includes disposing said micro-tube rack adjacent a stripper plate such that said at least one micro-tube is aligned with at least one stripper plate hole defined by said stripper plate.

32. The method of claim 30, wherein said at least one collet defines at least one collet pin cavity which traverses the length of said at least one collet.

33. The method of claim 32, wherein said configuring includes disposing at least one collet pin within said at least one collet pin cavity to cause said collet tip to be configured between said first tip width and said second tip width such that collet barbed portion securingly interacts with said micro-tube cap.

34. The method of claim 30, wherein said repositioning includes positioning said at least one collet such that said collet barbed portion exerts a force on said micro-tube cap directed away from said micro-tube.

35. The method of claim 32, wherein said removing said micro-tube cap includes removing said at least one collet pin from said at least one collet pin cavity to cause said collet tip to be configured between said second tip width and said first tip width.

36. The method of claim 30, wherein said removing includes collecting said micro-tube caps via a vacuum device.

37. A machine-readable computer program code, the program code including instructions for causing a controller to implement method for implementing a micro-tube handling device, wherein the micro-tube handling device includes at least one collet having a collet tip, wherein the collet tip includes a collet barbed portion and is sizably configurable between a first tip size and a second tip size, the method comprising:
associating a micro-tube rack adjacent the micro-tube handling device, wherein said micro-tube rack includes at least one micro-tube having a micro-tube cap which defines a cap cavity;
positioning said at least one collet adjacent said at least one micro-tube such that said collet barbed portion is at least partially disposed within said micro-tube cap cavity;
configuring said at least one collet tip between said first tip size and said second tip size to cause said collet barbed portion to securingly interact with said micro-tube cap;
repositioning said at least one collet away from said at least one micro-tube to cause said micro-tube cap to be removed from said micro-tube cavity; and
removing said micro-tube cap from said collet tip portion.

38. The machine-readable computer program code of claim 39, wherein the machine-readable computer program code is encoded onto a storage medium.
